(19) United States
(12) Patent Application Publication
(10) Pub. No.: US 2013/0177901 A1
(43) Pub. Date: Jul. 11, 2013

(54) MARKERS FOR THE PROGNOSIS AND RISK ASSESSMENT OF PREGNANCY-INDUCED HYPERTENSION AND PREECLAMPSIA

(71) Applicants: Bruno Darbouret, Tresques (FR); Gaiane Demirdjian, Nimes (FR)

(72) Inventors: Bruno Darbouret, Tresques (FR); Gaiane Demirdjian, Nimes (FR)

(73) Assignee: CEZANNE S.A.S., NIMES CEDEX 1 (FR)

(21) Appl. No.: 13/704,654

(22) PCT Filed: Jun. 17, 2011
(86) PCT No.: PCT/EP2011/003018

§ 371 Date: Feb. 28, 2013

(30) Foreign Application Priority Data
Jun. 18, 2010 (EP) 10290332.5

(51) Int. Cl. G01N 33/74; A61B 8/02

(52) U.S. Cl. CPC G01N 33/74; A61B 8/02
USPC 435/6.1; 436/501; 435/7.4; 600/437

(57) ABSTRACT

The present invention relates to the prognosis and risk assessment in pregnant women to develop pregnancy-induced hypertension and/or preeclampsia by the determination of marker levels.

### FIELD OF THE INVENTION

The present invention is in the field of clinical diagnostics. Particularly the present invention relates to the prognosis and risk assessment in pregnant women to develop pregnancy-induced hypertension and/or preeclampsia by the determination of marker levels.

### BACKGROUND OF THE INVENTION

Hypertension is the most common medical problem encountered during pregnancy, complicating 2-3% of pregnancies. Hypertensive disorders during pregnancy are classified into 4 categories, as recommended by the National High Blood Pressure Education Program Working Group on High Blood Pressure in Pregnancy: 1) chronic hypertension, 2) preeclampsia-eclampsia, 3) preeclampsia superimposed on chronic hypertension, and 4) gestational or pregnancy-induced hypertension (transient hypertension of pregnancy or chronic hypertension identified in the latter half of pregnancy). Chronic hypertension is defined as blood pressure exceeding 140/90 mm Hg before pregnancy or before 20 weeks' gestation. When hypertension is first identified during a woman's pregnancy and she is at less than 20 weeks' gestation, blood pressure elevations usually represent chronic hypertension. In contrast, new onset of elevated blood pressure readings after 20 weeks' gestation mandates the consideration and exclusion of preeclampsia. Preeclampsia occurs in up to 5% of all pregnancies, in 10% of first pregnancies, and in 20-25% of women with a history of chronic hypertension. Hypertensive disorders in pregnancy may cause maternal and fetal morbidity, and they remain a leading source of maternal mortality.

Gestational hypertension refers to hypertension with onset in the latter part of pregnancy (>20 weeks' gestation) without any other features of preeclampsia, and followed by normalization of the blood pressure postpartum. Of women who initially present with apparent gestational hypertension, about one third develops the syndrome of preeclampsia. As such, these patients should be observed carefully for this progression. The pathophysiology of gestational hypertension is unknown, but in the absence of features of preeclampsia, the maternal and fetal outcomes are usually normal. Gestational hypertension may, however, be a harbinger of chronic hypertension later in life.

Preeclampsia is a multi-system disorder in pregnancy, which is characterized by new onset of hypertension (systolic and diastolic blood pressure of ≧140 and 90 mm Hg, respectively) and proteinuria (protein excretion of ≧300 mg in a 24 h urine collection, or a dipstick of 2+), that develop after 20 weeks of gestation in a previously normotensive women (Magee et al. 2008. J Obstet Gynecol Canada 30 (3) Suppl 1:S1-S48). Dependent on the systemic involvement, several other symptoms, such as edema, disturbance of hemostasis, renal or liver failure, and the HELLP syndrome (hemolysis, elevated liver enzymes, and low platelet counts) also complicate the clinical picture. Preeclampsia can have an early onset (starting before 34 weeks of gestation) or late onset (starting after 34 weeks of gestation). Moreover, preeclampsia can show mild or severe symptoms (systolic blood pressure 160 mmHg or diastolic blood pressure ≧110 mmHg, proteinuria >5 g/24 hours, oliguria, neurological symptoms, other clinical symptoms such as deranged liver function, thrombocytopenia <100 000 mm³, HELLP syndrome), and can evolve in eclampsia in the most severe cases. In addition, it can manifest as a maternal disorder only, with an appropriate fetal growing, or it can present itself with a growth restricted fetus (in utero growth restriction (IUGR)) or sudden fetal distress. Preeclampsia is more common at the extremes of maternal age (<18 y or >35 y). The increased prevalence of chronic hypertension and other comorbid medical illnesses in women older than 35 years may explain the increased frequency of preeclampsia among older gravidas.

Although the exact pathophysiologic mechanism is not clearly understood, preeclampsia is primarily a disorder of placental dysfunction leading to a syndrome of endothelial dysfunction with associated vasospasm. In most cases, pathology demonstrates evidence of placental insufficiency with associated abnormalities such as diffuse placental thrombosis, an inflammatory placental decidual vasculopathy, and/or abnormal trophoblastic invasion of the endometrium. This supports abnormal placental development or placental damage from diffuse microthrombosis as being central to the development of this disorder.

Evidence also indicates that an altered maternal immune response to fetal/placental tissue may contribute to the development of preeclampsia.

The widespread endothelial dysfunction may manifest as a maternal syndrome, fetal syndrome, or both. The pregnant woman may manifest dysfunction of multiple organ systems, including the central nervous, hepatic, pulmonary, renal, and hematological systems. Endothelial damage leads to pathologic capillary leak that can present in the mother as rapid weight gain, nondependent edema (face or hands), pulmonary edema, hemoconcentration, or a combination thereof. The diseased placenta can also affect the fetus via decreased utero-placental blood flow. This decrease in perfusion can manifest clinically as nonreassuring fetal heart rate testing, low scores on a biophysical profile, oligohydramnios, or as fetal growth restriction.

Up to date, no therapeutic approaches are available for either treatment or prevention of preeclampsia, despite extensive clinical trials. Anti-hypertensive drugs, corticosteroids for lung maturation or magnesium sulfate to prevent from eclampsia are given to handle (or prevent the worsening of) the symptoms and can thus temporize over the short term to allow for safe delivery with a more mature fetus. As a consequence, the sole, though radical, resolution of preeclampsia is the removal of the placenta, and in case of prematurity, with the adverse consequence of delivering a pre-term baby. Therefore, preeclampsia, with or without IUGR, remains a major cause of maternal and neonatal mortality and morbidity worldwide.

Regardless of the lack of existing prophylactic and therapeutic means against preeclampsia, the search for noninvasive, biomarkers that could predict the development or assist in the detection of this life-threatening pregnancy disorder is still of utmost importance. Since many years, different biophysical and biochemical markers have been investigated, based on pathophysiological observations that have been noted in case of preeclampsia, such as placental dysfunction, a generalized inflammatory response, endothelial dysfunction and activation of the coagulation system (for review see Grill et al. 2009. Reproductive Biology and Endocrinology 7:70). These potential markers include angiogenic factors (e.g. VEGF, P1GF, sflt-1), soluble endoglin (sEng), P-selectin, cell-free fetal DNA, ADAM12, placental protein 13 (PP-13), Pentraxin 3 (PTX3) and pregnancy-associated plasma protein A (PAPP-A) (for review see Grill et al. 2009. Reproductive Biology and Endocrinology 7:70). Moreover, an imaging technique most widely used for predicting preeclampsia has been uteroplacental Doppler ultrasound. Impaired placental perfusion can be assessed by measuring flow waveform ratios or by detecting diastolic notching of the uterine arcuate vessels. However, it was shown that in both, low- and high-risk patient groups the predictive value was not sufficiently high to recommend routine screening (Conde-Agudelo et al. 2004. Obstet Gynecol 104: 1367-1391).

Adrenomedullin (ADM) and endothelin-1 (ET-1) are peptide hormones with vasoactive properties known to be present in the circulation. Both peptides are synthesized as larger prohormones and are released from their precursor peptides by proteolytical cleavage through peptide convertases. ADM and ET-1 were suggested to be implicated in the pathophysiology of hypertension (for review see: Murakami et al. 2006. Cardiovasc Hematol Disord Drug Targets 6(2): 125-132; Dhaun et al. 2008. Hypertension 52: 452-459).

The role of ADM for the diagnosis of preeclampsia has already been investigated with contradictory results. Senna et al. 2008 demonstrated that maternal circulating ADM values in patients with preeclampsia are different from those in normotensive pregnant women at different gestational ages (Senna et al. 2008. Medscape J Med 10(2):29). However, blood samples from pregnant women with preeclampsia were drawn after disease symptoms have already been manifested. Similarly Minegishi et al. 1999 measured ADM concentrations in plasma of non-pregnant, normal pregnant and pregnant women with preeclampsia. A gradual increase in plasma ADM was observed as pregnancy progressed. In the third trimester, plasma ADM concentrations did not differ significantly between women with and without pre-eclampsia (Minegishi et al. 1999. Mol Hum Reprod 5(8):767-70). Di Iorio et al. 1998 could show that pregnant women had higher adrenomedullin levels than nonpregnant subjects, although maternal plasma adrenomedullin concentrations did not differ between normal pregnant and preeclamptic women (Di Iorio et al. 1998. Hypertension 32(4): 758-63). In contrast, some studies could not demonstrate an increase but rather a decrease of ADM (Dikensoy et al. 2009 28(4):383-9; Hata et al. 1997. Lancet 350(9091):1600). However, none of these studies investigated the use of ADM as a marker for the prognosis or risk assessment of a pregnant woman to develop pregnancy-induced hypertension or preeclampsia.

The role of ET-1 for the diagnosis of preeclampsia has also been investigated in a number of studies and similarly to ADM with contradictory results. Baksu et al. 2005 and Nishikawa et al. 2000 showed that plasma ET-1 levels were significantly higher in the preeclampsia group than in healthy nonpregnant and normotensive pregnant women (Baksu et al. 2005. Int J Gynaecol Obstet 90(2):112-7; Nishikawa et al. 2000. Life Sci 67(12):1447-54). On the contrary, Zunker et al. 1998 showed that ET-1 levels were not statistically different between patients with pre-eclampsia and patients with normotensive uncomplicated pregnancy (Zunker et al. 1998. Fetal Diagn Ther. 13(5):309-14). However, none of these studies investigated the use of ET-1 as a marker for the prognosis or risk assessment of a pregnant woman to develop preeclampsia.

Gao et al. 1996 could detect that, compared with matched normal pregnant women, plasma ET-1 levels were significantly increased, in pregnancy-induced hypertension patients. Significant positive correlations existed between plasma ET-1 level and mean arterial pressure or the score index of the severity of PIH (Gao et al. 1996. Chin Med J (Engl). 109(11):823-6). Zhang et al. 1994 revealed that the levels of ET-1 in hypertensive pregnancy were higher than those of the normal pregnancy (Zhang et al. 1994. Zhonghua Fu Chan Ke Za Zh.29(11):645-7). However, in patients described in both Gao et al. and Zhang et al. ET-1 levels were measured at the time hypertension has already been manifested.

Thus, the inventors of the present invention have investigated whether the measurement of the levels of pro-ADM or fragments thereof and/or pro-ET-1 or fragments thereof, in particular, MR-pro-ADM and/or CT-pro-ET-1 levels, in a sample of a bodily fluid from a pregnant women could be used for the prognosis and risk assessment of pregnancy-induced hypertension and/or preeclampsia in these subjects.

### SUMMARY OF THE INVENTION

The present invention relates to a method for the prognosis of development of pregnancy-induced hypertension and/or preeclampsia or risk assessment in pregnant women to develop pregnancy-induced hypertension and/or preeclampsia comprising the steps of:
I. providing a sample of a bodily fluid of a subject,
II. determining the level of pro-ADM or fragments thereof and/or pro-ET-1 or fragments thereof in said sample,
III. correlating the level of pro-ADM or fragments thereof and/or pro-ET-1 or fragments thereof with the prognosis or risk assessment for a pregnant woman,
   wherein said fragments have a lengths of at least 6 amino acid residues.

### DESCRIPTION OF DRAWINGS

FIG. 1: Box plot analysis for MR-pro-ADM

FIG. 2: Box plot analysis for CT-pro-ET-1

FIG. 3: ROC plot analysis for MR-pro-ADM to differentiate between controls and patients who will develop a late-onset preeclampsia

FIG. 4: ROC plot analysis for CT-pro-ET-1 to differentiate between controls and patients who will develop a late-onset preeclampsia

FIG. 5: ROC plot analysis for CT-pro-ET-1 to differentiate between controls and patients who will develop a pregnancy induced hypertension (P1H)

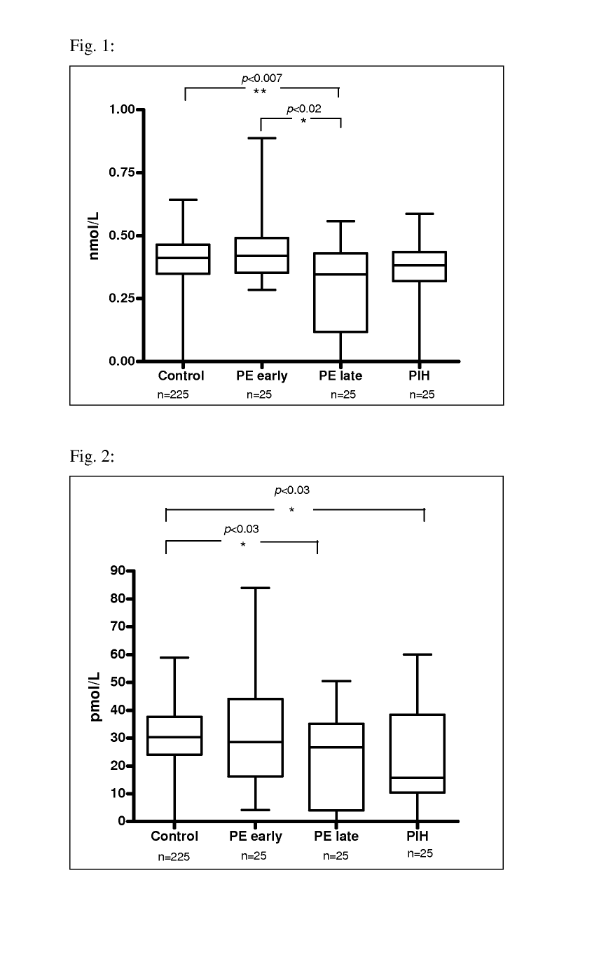

### DETAILED DESCRIPTION OF THE INVENTION

The present invention relates to a method for the prognosis of development of pregnancy-induced hypertension and/or preeclampsia or risk assessment in pregnant women to develop pregnancy-induced hypertension and/or preeclampsia comprising the steps of:
I. providing a sample of a bodily fluid of a subject,
II. determining the level of pro-ADM or fragments thereof and/or pro-ET-1 or fragments thereof in said sample,
III. correlating the level of pro-ADM or fragments thereof and/or pro-ET-1 or fragments thereof with the prognosis or risk assessment for a pregnant woman,
   wherein said fragments have a lengths of at least 6 amino acid residues.

Said fragments have preferable a length of at least 6 amino acids, more preferably a length of at least 12 amino acid residues. Such fragments are preferably detectable with immunological assays as described herein.

According to the present invention a decrease of the level of pro-ADM or fragments thereof and/or pro-ET-1 or fragments thereof is indicative for an enhanced risk of pregnancy-induced hypertension and/or preeclampsia when compared with the level of pro-ADM or fragments thereof and/or pro-ET-1 or fragments thereof in sample from subjects not having a risk of pregnancy-induced hypertension and/or preeclampsia.

In one embodiment of the invention the measurement of pro-ADM or fragments thereof and/or pro-ET-1 or fragments thereof is carried out within the first to second trimester (8^{th} to 26^{th} week of pregnancy), more preferred within the first to early second trimester (8^{th} to 20^{th} week of pregnancy), even more preferred within the first trimester (8^{th} to 14^{th} week of pregnancy), mostly preferred within the early first trimester (8^{th} to 10^{th} week of pregnancy). In one embodiment the invention the measurement of pro-ADM or fragments thereof and/or pro-ET-1 or fragments thereof is carried out before 25^{th} week, preferably between 8^{th} and 24^{th} week of pregnancy.

In a further embodiment of the invention the prognosis is related to an early onset (between 20 to 34 weeks of gestation) or a late onset (after 34 weeks of gestation) of preeclampsia.

In another embodiment of the invention further markers may additionally be determined selected from the group sflt-1, sEng, PIGF, VEGF, PP-13, ADAM12, P-Selectin, cell-free fetal DNA, PTX3, PAPP-A, visfatin, inhibin A, activin A, human chorionic gonadotropin (hCG), beta-hCG, alpha-fetoprotein (AFP), metalloproteinase-9 (MMP-9), ultrasound markers (uterine artery pulsatility index and/or diastolic notching) as well as pro-atrial natriuretic peptide (pro-ANP) or fragments thereof, pro-brain natriuretic peptide (pro-BNP) or fragments thereof and pro-Vasopres sin or fragments thereof.

In a preferred embodiment of the invention said further markers are selected from the group comprising sflt-1, sEng, PIGF, VEGF, PP-13, ADAM12, P-Selectin, cell-free fetal DNA, PTX3, PAPP-A, visfatin, inhibin A, activin A, hCG, beta-hCG, AFP, MMP-9, ultrasound markers (uterine artery pulsatility index and/or diastolic notching), MR-proANP, NT-proBNP and Copeptin.

The invention also relates to the use of the described methods and kits for the prognosis and risk assessment of pregnancy-induced hypertension and/or preeclampsia in pregnant women.

The term “pregnancy-induced hypertension” or gestational hypertension is defined as the development of new arterial hypertension in a pregnant woman after 20 weeks of gestation (systolic and diastolic blood pressure of ≧140 and 90 mm Hg, respectively).

The term “preeclampsia” includes a hypertensive, multi-system disorder of pregnant women, characterized by hypertension and proteinuria. The most common symptoms of preeclampsia are high blood pressure, increased protein in the urine, and swelling or edema of hands and face. In certain embodiments of the invention, preeclampsia is defined as hypertension (systolic and diastolic blood pressure of ≧140 and 90 mm Hg, respectively) and proteinuria (protein excretion of ≧300 mg in a 24 h urine collection, or a dipstick of ≧2+).

In a particular embodiment of the invention the pregnancy-induced hypertension and/or preeclampsia is asymptomatic and/or is not manifested at the time of measuring. Thus, asymptomatic and/or not manifested means systolic and diastolic blood pressure of less than 140 and 90 mm Hg and/or protein excretion of less than 300 mg in a 24 h urine collection, or a dipstick of less than 2+.

“Prognosis” relates to the prediction of an outcome or a specific risk for a subject suffering from a particular disease or clinical condition. This may include an estimation of the chance of recovery or the chance of an adverse outcome for said subject.

The term “sample” as used herein refers to a sample of bodily fluid obtained for the purpose of diagnosis, prognosis, or evaluation of a subject of interest, such as a patient. Preferred test samples include blood, serum, plasma, cerebrospinal fluid, urine, saliva, sputum, and pleural effusions. In addition, one of skill in the art would realize that some test samples would be more readily analyzed following a fractionation or purification procedure, for example, separation of whole blood into serum or plasma components.

Thus in a preferred embodiment of the invention the sample is selected from the group consisting of a blood sample, a serum sample, a plasma sample, a cerebrospinal fluid sample, a saliva sample and an urine sample or an extract of any of the aforementioned samples. Preferably, the sample is a blood sample, most preferably a serum sample or a plasma sample.

The term “subject” as used herein refers to a living human or non-human organism. Preferably herein the subject is a human subject that is pregnant within the first to second trimester (8^{th} to 26^{th} week of pregnancy), more preferred within the first to second trimester (8^{th} to 24^{th} week of pregnancy), even more preferred within the first to early second trimester (8^{th} to 20^{th} week of pregnancy), even more preferred within the first trimester (8^{th} to 14^{th} week of pregnancy), mostly preferred within the early first trimester (8^{th} to 10^{th} week of pregnancy), In another preferred embodiment the subject is a human subject that is pregnant within before 25^{th} week, preferably between 8^{th} and 24^{th} week of pregnancy.

The term “correlating”, as used herein in reference to the use of diagnostic and prognostic marker(s), refers to comparing the presence or amount of the marker(s) in a patient to its presence or amount in persons known to suffer from, or known to be at risk of, a given condition. A marker level in a patient sample can be compared to a level known to be associated with a specific diagnosis. The sample's marker level is said to have been correlated with a diagnosis; that is, the skilled artisan can use the marker level to determine whether the patient suffers from a specific type diagnosis, and respond accordingly. Alternatively, the sample's marker level can be compared to a marker level known to be associated with a good outcome (e.g. the absence of disease etc.). In preferred embodiments, a panel of marker levels is correlated to a global probability or a particular outcome.

As mentioned herein in the context of proteins or peptides, the term “fragment” refers to smaller proteins or peptides derivable from larger proteins or peptides, which hence comprise a partial sequence of the larger protein or peptide. Said fragments are derivable from the larger proteins or peptides by saponification of one or more of its peptide bonds.

The term “level” in the context of the present invention relates to the concentration (preferably expressed as weight/volume; w/v) of marker peptides taken from a sample of a patient.

Determining the level of pro-ADM or fragments thereof and/or pro-ET-1 or fragments thereof herein is performed using a detection method and/or a diagnostic assay. A preferred pro-ADM fragment is MR-pro-ADM. A preferred pro-ET-1 fragment is CT-pro-ET-1.

MR-pro-ADM has the following sequence:

| SEQ ID No. 1: | |
|---|---|
| 1 | ELRMSSSYPT GLADVKAGPA QTLIRPQDMK GASRSPEDSS |

CT-pro-ET-1 has the following sequence:

SEQ ID No. 2:

| | |
|---|---|
| 1 | RSSEEHLRQT RSETMRNSVK SSFHDPKLKG KPSRERYVTH |
| | NRAHW |

As mentioned herein, an “assay” or “diagnostic assay” can be of any type applied in the field of diagnostics. Such an assay may be based on the binding of an analyte to be detected to one or more capture probes with a certain affinity. Concerning the interaction between capture molecules and target molecules or molecules of interest, the affinity constant is preferably greater than 10⁸ M⁻¹.

In the context of the present invention, “capture molecules” are molecules which may be used to bind target molecules or molecules of interest, i.e. analytes (i.e. in the context of the present invention the cardiovascular peptide(s)), from a sample. Capture molecules must thus be shaped adequately, both spatially and in terms of surface features, such as surface charge, hydrophobicity, hydrophilicity, presence or absence of lewis donors and/or acceptors, to specifically bind the target molecules or molecules of interest. Hereby, the binding may for instance be mediated by ionic, van-der-Waals, pi-pi, sigma-pi, hydrophobic or hydrogen bond interactions or a combination of two or more of the aforementioned interactions between the capture molecules and the target molecules or molecules of interest. In the context of the present invention, capture molecules may for instance be selected from the group comprising a nucleic acid molecule, a carbohydrate molecule, a RNA molecule, a protein, an antibody, a peptide or a glycoprotein. Preferably, the capture molecules are antibodies, including fragments thereof with sufficient affinity to a target or molecule of interest, and including recombinant antibodies or recombinant antibody fragments, as well as chemically and/or biochemically modified derivatives of said antibodies or fragments derived from the variant chain with a length of at least 12 amino acids thereof.

The preferred detection methods comprise immunoassays in various formats such as for instance radioimmunoassay (RIA), chemiluminescence- and fluorescence-immunoassays, Enzyme-linked immunoassays (ELISA), Luminex-based bead arrays, protein microarray assays, and rapid test formats such as for instance immunochromatographic strip tests.

The assays can be homogenous or heterogeneous assays, competitive and non-competitive assays. In a particularly preferred embodiment, the assay is in the form of a sandwich assay, which is a non-competitive immunoassay, wherein the molecule to be detected and/or quantified is bound to a first antibody and to a second antibody. The first antibody may be bound to a solid phase, e.g. a bead, a surface of a well or other container, a chip or a strip, and the second antibody is an antibody which is labeled, e.g. with a dye, with a radioisotope, or a reactive or catalytically active moiety. The amount of labeled antibody bound to the analyte is then measured by an appropriate method. The general composition and procedures involved with “sandwich assays” are well-established and known to the skilled person (The Immunoassay Handbook, Ed. David Wild, Elsevier LTD, Oxford; 3rd ed. (May 2005), ISBN-13: 978-0080445267; Hultschig C et al., Curr Opin Chem. Biol. 2006 February; 10(1):4-10. PMID: 16376134, incorporated herein by reference).

In a particularly preferred embodiment the assay comprises two capture molecules, preferably antibodies which are both present as dispersions in a liquid reaction mixture, wherein a first labeling component is attached to the first capture molecule, wherein said first labeling component is part of a labeling system based on fluorescence- or chemiluminescence-quenching or amplification, and a second labeling component of said marking system is attached to the second capture molecule, so that upon binding of both capture molecules to the analyte a measurable signal is generated that allows for the detection of the formed sandwich complexes in the solution comprising the sample.

Even more preferred, said labeling system comprises rare earth cryptates or rare earth chelates in combination with fluorescence dye or chemiluminescence dye, in particular a dye of the cyanine type.

In the context of the present invention, fluorescence based assays comprise the use of dyes, which may for instance be selected from the group comprising FAM (5- or 6-carboxyfluorescein), VIC, NED, Fluorescein, Fluoresceinisothiocyanate (FITC), IRD-700/800, Cyanine dyes, such as CY3, CY5, CY3.5, CY5.5, Cy7, Xanthen, 6-Carboxy-2′,4′,7′,4,7-hexachlorofluorescein (HEX), TET, 6-Carboxy-4′,5′-dichloro-2′,7′-dimethody-fluorescein (JOE), N,N,N′,N′-Tetramethyl-6-carboxyrhodamine (TAMRA), 6-Carboxy-X-rhodamine (ROX), 5-Carboxyrhodamine-6G (R6G5), 6-carboxyrhodamine-6G (RG6), Rhodamine, Rhodamine Green, Rhodamine Red, Rhodamine 110, BODIPY dyes, such as BODIPY TMR, Oregon Green, Coumarines such as Umbelliferone, Benzimides, such as Hoechst 33258; Phenanthridines, such as Texas Red, Yakima Yellow, Alexa Fluor, PET, Ethidiumbromide, Acridinium dyes, Carbazol dyes, Phenoxazine dyes, Porphyrine dyes, Polymethin dyes, and the like. In the context of the present invention, chemiluminescence based assays comprise the use of dyes, based on the physical principles described for chemiluminescent materials (Kirk-Othmer, Encyclopedia of chemical technology, 4th ed., executive editor, J. I. Kroschwitz; editor, M. Howe-Grant, John Wiley & Sons, 1993, vol. 15, p. 518-562, incorporated herein by reference, including citations on pages 551-562). Preferred chemiluminescent dyes are acridiniumesters.

The sensitivity and specificity of a diagnostic and/or prognostic test depends on more than just the analytical “quality” of the test, they also depend on the definition of what constitutes an abnormal result. In practice, Receiver Operating Characteristic curves (ROC curves), are typically calculated by plotting the value of a variable versus its relative frequency in “normal” (i.e. apparently healthy) and “disease” populations (i.e. patients suffering from diabetes, insulin resistance and/or metabolic syndrome). For any particular marker, a distribution of marker levels for subjects with and without a disease will likely overlap. Under such conditions, a test does not absolutely distinguish normal from disease with 100% accuracy, and the area of overlap indicates where the test cannot distinguish normal from disease. A threshold is selected, above which (or below which, depending on how a marker changes with the disease) the test is considered to be abnormal and below which the test is considered to be normal. The area under the ROC curve is a measure of the probability that the perceived measurement will allow correct identification of a condition. ROC curves can be used even when test results don't necessarily give an accurate number. As long as one can rank results, one can create a ROC curve. For example, results of a test on “disease” samples might be ranked according to degree (e.g. 1=low, 2=normal, and 3=high). This ranking can be correlated to results in the “normal” population, and a ROC curve created. These methods are well known in the art (See, e.g., Hanley et al. 1982. Radiology 143: 29-36). Preferably, a threshold is selected to provide a ROC curve area of greater than about 0.5, more preferably greater than about 0.7, still more preferably greater than about 0.8, even more preferably greater than about 0.85, and most preferably greater than about 0.9. The term “about” in this context refers to +/−5% of a given measurement.

The horizontal axis of the ROC curve represents (1-specificity), which increases with the rate of false positives. The vertical axis of the curve represents sensitivity, which increases with the rate of true positives. Thus, for a particular cut-off selected, the value of (1-specificity) may be determined, and a corresponding sensitivity may be obtained. The area under the ROC curve is a measure of the probability that the measured marker level will allow correct identification of a disease or condition. Thus, the area under the ROC curve can be used to determine the effectiveness of the test.

In certain embodiments, markers and/or marker panels are selected to exhibit at least about 70% sensitivity, more preferably at least about 80% sensitivity, even more preferably at least about 85% sensitivity, still more preferably at least about 90% sensitivity, and most preferably at least about 95% sensitivity, combined with at least about 70% specificity, more preferably at least about 80% specificity, even more preferably at least about 85% specificity, still more preferably at least about 90% specificity, and most preferably at least about 95% specificity. In particularly preferred embodiments, both the sensitivity and specificity are at least about 75%, more preferably at least about 80%, even more preferably at least about 85%, still more preferably at least about 90%, and most preferably at least about 95%. The term “about” in this context refers to +/−5% of a given measurement.

According to the method, the development of a pregnancy-induced hypertension and/or preeclampsia is predicted in a pregnant woman when said determined level of MR-pro-ADM is lower than a predetermined threshold level. Preferably, the predetermined threshold level of MR-pro-ADM is between 0.2 and 0.6 nmol/L, more preferably between 0.2 nmol/L and 0.5 nmol/L, even more preferred between 0.2 nmol/L and 0.4 nmol/L, most preferred between 0.2 nmol/L and 0.3 nmol/L. In a preferred embodiment the development of a pregnancy-induced hypertension and/or preeclampsia is predicted in a pregnant woman when said determined level of MR-pro-ADM or fragments thereof is lower than 0.6 nmol/L, preferably lower than 0.5 nmol/L, more preferably lower than 0.4 nmol/L, most preferred lower than 0.3 nmol/L.

According to the method, the development of a pregnancy-induced hypertension and/or preeclampsia is predicted in a pregnant woman when said determined level of CT-pro-ET-1 or fragments thereof is lower than a predetermined threshold level. Preferably, the predetermined threshold level of CT-pro-ET-1 or fragments thereof is between 20 and 60 pmol/L, more preferably between 20 pmol/L and 50 pmol/L, even more preferred between 20 pmol/L and 40 pmol/L, most preferred between 20 pmol/L and 30 pmol/L. In a preferred embodiment the development of a pregnancy-induced hypertension and/or preeclampsia is predicted in a pregnant woman when said determined level of CT-pro-ET-1 or fragments thereof is lower than 60 pmol/L, preferably lower than 50 pmol/L, more preferably lower than 40 pmol/L, most preferred lower than 30 pmol/L.

### EXAMPLES

### Study Population

A total of 323 patients were included into the retrospective study. These patients were diagnosed to suffer from early-onset preeclampsia (n=25), late-onset preeclampsia (n=25) and pregnancy induced hypertension (P1H) (n=25). 225 pregnant women without these diseases served as controls.

EDTA-samples were taken at the time of each prenatal visit, which is held at 11 to 14 weeks of gestation. At that time all patients included into the study were asymptomatic and did not show any signs or symptoms for preeclampsia or PIH. All pregnant women signed a consent form approved by King's College Hospital Ethics Committee.

A patient was diagnosed to suffer from preeclampsia if hypertension (systolic or diastolic blood pressure of ≧140 and 90 mm Hg, respectively) and proteinuria (protein excretion of >300 mg in a 24 h urine collection, or a dipstick of ≧2+) was detected after 20 weeks of gestation. Patients with the diagnosis of preeclampsia were further classified according to the time of preeclampsia onset as early-onset preeclampsia (onset of symptoms between week 20 and 34 of gestation) and late-onset preeclampsia (onset of symptoms after 34 weeks of gestation).

A patient was diagnosed to suffer from PIH if the diastolic blood pressure of ≧90 mm Hg was detected on ≧2 occasions 4 hours apart after 20 weeks of gestation in previously normotensive women in the absence of significant proteinuria.

### Measurements

MR-pro-ADM and CT-pro-ET-1 were detected using novel fully automated sandwich immunoassay systems on the B.R.A.H.M.S KRYPTOR (B.R.A.H.M.S GmbH, Hennigsdorf/Berlin, Germany) (Caruhel et al. 2009. Clin Biochem 42:725-8). This random access analyzer employs the sensitive Time Resolved Amplified Cryptate Emission (TRACE) technology, based on a non-radioactive-transfer between 2 fluorophores, europium cryptate and XL665. The automated assay for the detection of MR-pro-ADM is based essentially on the sandwich fluorescence assay using the same antibody pair as described in detail elsewhere (Morgenthaler et al. 2005 Clin Chem 51:1823-9). For MR-pro-ADM detection, 26 μl plasma was incubated for 29 min. The measuring range was 0-100 nmol/L, the limit of detection and limit of quantification were 0.05 and 0.23 nmol/L, respectively. The intra assay CV was 1.9% and the inter laboratory CV was 9.8% at 1.17 nmol/L.

The automated sandwich fluorescence assay for the detection of CT-proET-1 uses a mouse monoclonal antibody directed against a peptide comprising the amino acids 167 to 183 of the human pro-ET-1 sequence (SEQ. ID No. 3) and a sheep polyclonal antibody directed against a peptide comprising the amino acids 183 to 195 of the human pro-ET-1 sequence (SEQ. ID No. 4). For CT-proET-1 detection, 50 μl plasma was incubated for 24 min. The measuring range of the assay was 0-500 pmol/L, the limit of detection and limit of quantification were 2.8 and 9.78 pmol/L, respectively. The intra- and inter-assay CV determined in the range of 44-324 pmol/L were 1.3-4.6% and 6.3-9.6%, respectively (Caruhel et al. 2008 AACC 54:6, Supplement A119/C-63, abstract).

### Results

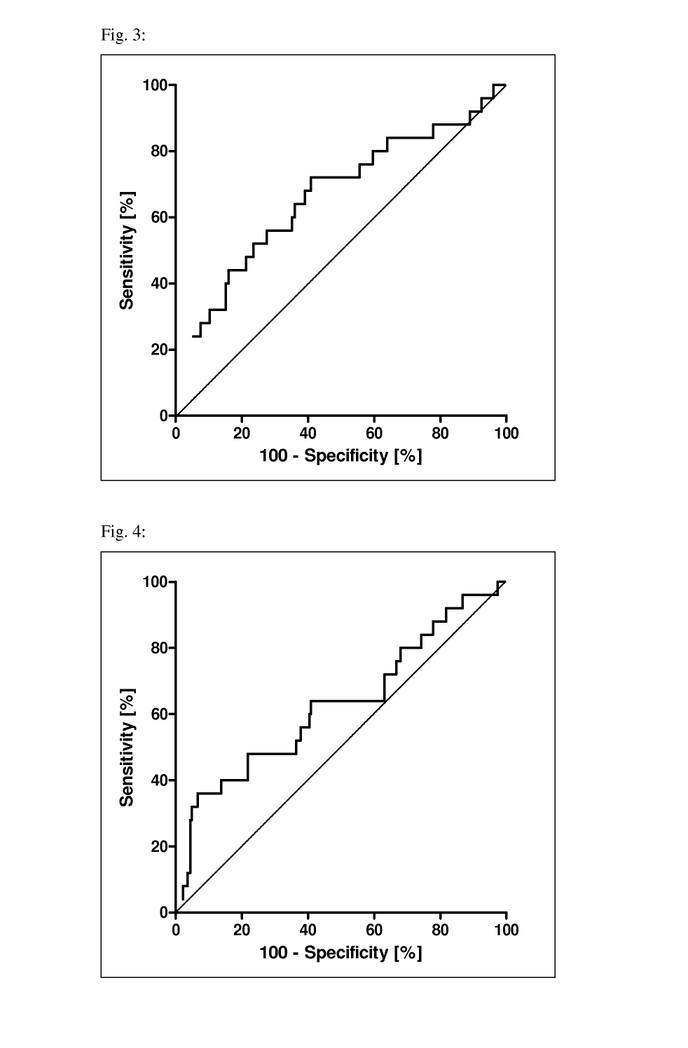

The patients' characteristics are shown in table 1. Median MR-pro-ADM levels were significantly lower in women who developed a late-onset preeclampsia when compared to pregnant controls (p<0.007) or women who developed an early-onset of preeclampsia (p<0.02). There was a tendency towards lower MR-pro-ADM concentrations in the PIH group, although not statistically significant. The area under the ROC curve (AUC) to differentiate between pregnant controls and women who will develop a late-onset preeclampsia was 0.66 (p<0.007) for MR-pro-ADM (see FIG. 3). The sensitivities and specificities of exemplary MR-pro-ADM cut-off values are given in table 2.

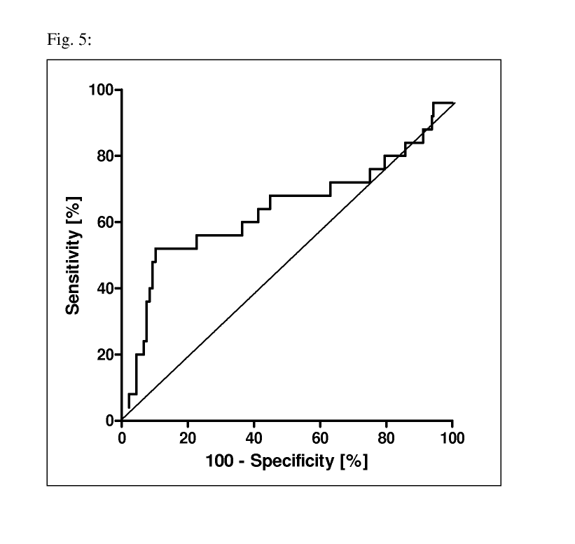

Median CT-pro-ET-1 levels were significantly lower in women who developed a late-onset preeclampsia when compared to pregnant controls (p<0.03). Moreover, the CT-pro-ET-1 concentration was significantly lower in women who developed pregnancy-induced hypertension (p<0.03) in comparison to pregnant non-hypertensive controls. For CT-pro-ET-1 the area under the ROC curve (AUC) to differentiate between pregnant controls and women who will develop a late-onset preeclampsia was 0.63 (p<0.05) (see FIG. 4). The sensitivities and specificities of exemplary CT-pro-ET-1 cut-off values to differentiate between pregnant non-hypertensive controls and women who will develop late-onset preeclampsia are given in table 3. The AUC for CT-pro-ET-1 to differentiate between pregnant non-hypertensive controls and women who will develop PIH was 0.64 (p<0.03) (see FIG. 5). The sensitivities and specificities of exemplary CT-pro-ET-1 cut-off values to differentiate between pregnant non-hypertensive controls and women who will develop PIH are given in table 4.

### Sequences

| SEQ ID No. 1: | |
|---|---|
| 1 | ELRMSSSYPT GLADVKAGPA QTLIRPQDMK GASRSPEDSS |
| SEQ ID No. 2: | |
| 1 | RSSEEHLRQT RSETMRNSVK SSFHDPKLKG KPSRERYVTH |
| | NRAHW |
| SEQ ID No. 3: | |
| 1 | NSVKSSFHDPKLKGKPS |
| SEQ ID No. 4: | |
| 1 | SRERYVTHNRAHW |

TABLE 1
Patient characteristics

| | | Preeclampsia | Preeclampsia | |
|---|---|---|---|---|
| Diagnosis | control | (early onset) | (late onset) | PIH |
| N | 225 | 25 | 25 | 25 |
| Mean maternal age | 31.2 | 33.7 | 30.8 | 34.6 |
| MR-pro-ADM | 0.41 | 0.42 | 0.35 | 0.38 |
| (median in nmol/L) | | | | |
| CT-pro-ET-1 | 30.4 | 28.5 | 26.7 | 15.8 |
| (median in pmol/L) | | | | |

TABLE 2
Specificity and sensitivity values at different cut-off levels for
MR-pro-ADM to differentiate between pregnant
controls and women who will develop a late-onset preeclampsia

| MR-pro-ADM cut-off value | Specificity | Sensitivity |
|---|---|---|
| (nmol/L) | (in %) | (in %) |
| 0.32 | 84 | 44 |
| 0.35 | 76 | 52 |
| 0.38 | 64 | 64 |
| 0.40 | 56 | 72 |

TABLE 3
Specificity and sensitivity values at different cut-off levels for CT-pro-ET-1
to differentiate between pregnant normotensive controls and women
who will develop a late-onset preeclampsia

| CT-pro-ET-1 cut-off value | Specificity | Sensitivity |
|---|---|---|
| (pmol/L) | (in %) | (in %) |
| 18.5 | 86 | 40 |
| 23.0 | 78 | 48 |
| 28.2 | 59 | 64 |
| 33.5 | 37 | 72 |

TABLE 4
Specificity and sensitivity values at different cut-off levels
for CT-pro-ET-1 to differentiate between pregnant
normotensive controls and women who will PIH

| CT-pro-ET-1 cut-off value | Specificity | Sensitivity |
|---|---|---|
| (pmol/L) | (in %) | (in %) |
| 15.8 | 90 | 52 |
| 26.8 | 63 | 60 |
| 29.1 | 55 | 68 |
| 33.5 | 37 | 72 |

1. A method for the prognosis of development of pregnancy-induced hypertension and/or preeclampsia or risk assessment in pregnant women to develop pregnancy-induced hypertension and/or preeclampsia comprising the steps of:
(i) providing a sample of a bodily fluid of a subject,
(ii) determining the level of pro-ADM or fragments thereof and/or pro-ET-1 or fragments thereof in said sample,
(iii) correlating the level of pro-ADM or fragments thereof and/or pro-ET-1 or fragments thereof with the prognosis or risk assessment for a pregnant woman,
wherein said fragments have a lengths of at least 6 amino acid residues.

2. The method of claim 1, wherein the determination of the level pro-ADM or fragments thereof and/or pro-ET-1 or fragments thereof is carried out within the first to second trimester (8^{th} to 26^{th} week of pregnancy), more preferred within the first to early second trimester (8^{th} to 20^{th} week of pregnancy), even more preferred within the first trimester (8^{th} to 14^{th} week of pregnancy), mostly preferred within the early first trimester (8^{th} to 10^{th} week of pregnancy).

3. The method of claim 1, wherein the prognosis or risk assessment is related to an early onset (between 20 to 34 weeks of gestation) or a late onset (after 34 weeks of gestation) of preeclampsia.

4. The method of claim 1, wherein at least one further marker selected from the group sflt-1, sEng, PIGF, VEGF, PP-13, ADAM12, P-Selectin, cell-free fetal DNA, PTX3, PAPP-A, Visfatin, inhibin A, activin A, human chorionic gonadotropin (hCG), alpha-fetoprotein (AFP), metalloproteinase-9 (MMP-9), ultrasound markers (uterine artery pulsatility index and/or diastolic notching), pro-atrial natriuretic peptide (proANP) or fragments thereof, pro-brain natriuretic peptide (proBNP) or fragments thereof and pro-Vasopressin or fragments thereof is determined.

5. The method of claim 1, wherein the level of MR-pro-ADM and/or CT-pro-ET-1 is determined.

6. The method of claim 1, wherein the determination of the level of MR-pro-ADM and/or CT-pro-ET-1 is combined with PAPP-A, PLGF and/or ultrasound markers (uterine artery pulsatility index and/or diastolic notching).

7. The method of claim 1, wherein pregnancy-induced hypertension and/or preeclampsia is asymptomatic and/or not manifested in said pregnant woman at the time of measuring.

8. The method of claim 1, wherein said sample is a bodily fluid, in particular blood, serum, plasma, cerebrospinal fluid, urine, saliva or a pleural effusion.

9. The method of claim 1, wherein the predetermined threshold level of MR-pro-ADM is between 0.2 and 0.6 nmol/L, more preferably between 0.2 nmol/L and 0.5 nmol/L, even more preferred between 0.2 nmol/L and 0.4 nmol/L, most preferred between 0.2 nmol/L and 0.3 nmol/L.

10. The method of claim 1, wherein the predetermined threshold level of CT-pro-ET-1 is between 20 and 60 pmol/L, more preferably between 20 pmol/L and 50 pmol/L, even more preferred between 20 pmol/L and 40 pmol/L, most preferred between 20 pmol/L and 30 pmol/L.

11. The method of claim 1, wherein the development of a pregnancy-induced hypertension and/or preeclampsia is predicted in a pregnant woman when said determined level of MR-pro-ADM or CT-pro-ET-1 is lower than the predetermined threshold level.


SEQUENCE LISTING (4 sequences)

SEQ ID NO: 1 - protein, 48 aa, Homo sapiens
ELRMSSSYPTGLADVKAGPAQTLIRPQDMKGASRSPEDSSPDAARIRV

SEQ ID NO: 2 - protein, 45 aa, Homo sapiens
RSSEEHLRQTRSETMRNSVKSSFHDPKLKGKPSRERYVTHNRAHW

SEQ ID NO: 3 - protein, 17 aa, Homo sapiens
NSVKSSFHDPKLKGKPS

SEQ ID NO: 4 - protein, 13 aa, Homo sapiens
SRERYVTHNRAHW